(19) United States
(12) Patent Application Publication
(10) Pub. No.: US 2024/0415624 A1
(43) Pub. Date: Dec. 19, 2024

(54) TAIL VEIN INJECTION SYSTEM, TAIL VEIN INJECTION METHOD, AND NON-TRANSITORY COMPUTER-READABLE STORAGE MEDIUM

(71) Applicant: Preferred Networks, Inc., Tokyo (JP)

(72) Inventors: Tianyi TESHIMA, Tokyo (JP); Koji TERADA, Tokyo (JP); Koichi NISHIWAKI, Tokyo (JP); Shun MITSUMATA, Tokyo (JP); Hidetomo KITAMURA, Tokyo (JP)

(21) Appl. No.: 18/819,274

(22) Filed: Aug. 29, 2024

Related U.S. Application Data
(63) Continuation of application No. PCT/JP2023/007139, filed on Feb. 27, 2023.

(30) Foreign Application Priority Data
Mar. 2, 2022 (JP) 2022-032006

(51) Int. Cl. A61D 1/02; A61M 5/32; A61M 5/46

(52) U.S. Cl. CPC A61D 1/025; A61M 5/3245; A61M 5/46

(57) ABSTRACT

A tail vein injection system includes at least one memory; and at least one processor connected to the at least one memory. The at least one processor is configured to: calculate information including a position of a tail vein based on one or more images of a tail; adjust a position and a posture of a syringe needle based on the calculated information; and after the adjusting, perform a puncture of the tail vein using the syringe needle.

### CROSS-REFERENCE TO RELATED APPLICATIONS

The present application is a continuation application of International Application No. PCT/JP2023/007139, filed on Feb. 27, 2023, which claims priority to Japanese Patent Application No. 2022-032006, filed on Mar. 2, 2022. The contents of these applications are incorporated herein by reference in their entirety.

### BACKGROUND

### 1. Field of the Invention

The present disclosure relates to a tail vein injection system, a tail vein injection method, and a non-transitory computer-readable storage medium.

### 2. Description of the Related Art

Animal experiments play an important role in various fields, such as biochemistry, drug discovery, and the like. Especially, laboratory mice are widely used as laboratory animals. When mice are punctured, tail vein injection may be performed.

As a technique related to puncturing the blood vessel of a human arm, an automated injection device is known (see, for example, Japanese Laid-Open Patent Application Publication No. 2019-030570). This automated injection device is configured to obtain first and second images captured by first and second cameras, represent characteristic shapes of sites of the blood vessel with a first-degree linear formula from the first and second images, and control an actuator to puncture the blood vessel by a puncture needle of a syringe.

### SUMMARY

A tail vein injection system according to one embodiment of the present disclosure includes at least one memory; and at least one processor connected to the at least one memory. The at least one processor is configured to: calculate information including a position of a tail vein based on one or more images of a tail; adjust a position and a posture of a syringe needle based on the calculated information; and after the adjusting, perform a puncture of the tail vein using the syringe needle.

### BRIEF DESCRIPTION OF THE DRAWINGS

FIG. 1 is a schematic diagram of a tail vein injection system according to an embodiment of the present disclosure.

FIG. 2A is a diagram (1) illustrating an image of a tail vein injection method according to the embodiment.

FIG. 2B is a diagram (2) illustrating an image of the tail vein injection method according to the embodiment.

FIG. 3 is a diagram illustrating configuration examples of a bent jig and a retainer according to the embodiment.

FIG. 4 is a diagram illustrating a configuration example of a clamp according to the embodiment.

FIG. 5 is a diagram illustrating an example of a functional configuration of an information processing device according to the embodiment.

FIG. 6A is a diagram (1) describing a detection process of a tail vein according to the embodiment.

FIG. 6B is a diagram (2) describing the detection process of the tail vein according to the embodiment.

FIG. 7A is a diagram (1) describing a detection process of a needle of a syringe according to the embodiment.

FIG. 7B is a diagram (2) describing the detection process of the needle of the syringe according to the embodiment.

FIG. 8 is a flowchart illustrating a flow of a process of the tail vein injection system according to the embodiment.

FIG. 9 is a flowchart illustrating an example of a calculation process according to the embodiment.

FIG. 10 is a diagram (1) describing the calculation process according to the embodiment.

FIG. 11 is a diagram (2) describing the calculation process according to the embodiment.

FIG. 12 is a diagram (3) describing the calculation process according to the embodiment.

FIG. 13 is a diagram (4) describing the calculation process according to the embodiment.

FIG. 14 is a diagram (5) describing the calculation process according to the embodiment.

FIG. 15 is a diagram illustrating an example of a hardware configuration of the information processing device according to the embodiment.

### DETAILED DESCRIPTION

The diameter of the tail vein of a mouse is about 300 micrometers (μm), which is about 1/10 the diameter of the blood vessel of a human arm. Therefore, it may be challenging to apply the technique of puncturing the blood vessel of the human arm, as described in Japanese Laid-Open Patent Application Publication No. 2019-030570, to the tail vein injection.

An embodiment of the present disclosure provides a tail vein injection system that facilitates the puncture of the tail vein.

Hereinafter, embodiments of the present disclosure will be described in detail with reference to the drawings. In the following description, components common across the drawings are denoted by the same symbols, and description thereof will be omitted. For ease of understanding, the scale of each of the components in the drawings may be different from the actual scale thereof. An X-axis direction, a Y-axis direction, and a Z-axis direction include a direction parallel to an X axis, a direction parallel to a Y axis, and a direction parallel to a Z axis, respectively. The X-axis direction, the Y-axis direction, and the Z-axis direction are orthogonal to each other. Note that, in the following description, such terms as parallel, orthogonal, vertical, horizontal, direction, and the like permit deviation to such an extent that the effects of the present disclosure are not impaired.

### <System Configuration>

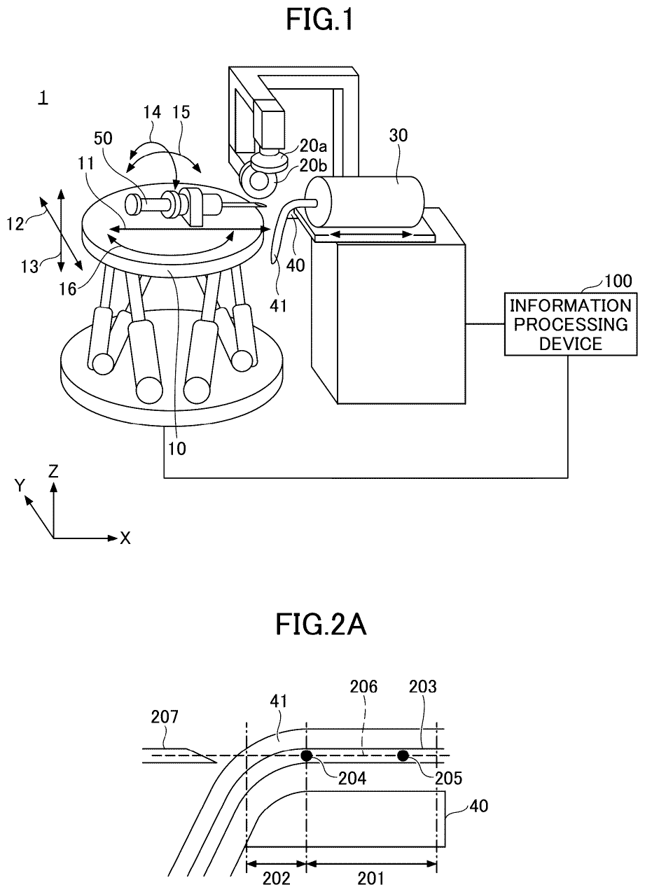

FIG. 1 is a schematic diagram of a tail vein injection system according to an embodiment of the present disclosure. A tail vein injection system 1 includes, for example: a position adjuster 10 having five or more axes; a first camera 20a; a second camera 20b; a retainer 30 configured to retain a mouse or the like; a fixture 40 configured to retain a tail 41 of a mouse or the like; a syringe 50; and an information processing device 100.

The position adjuster 10 is, for example, a robot having six axial degrees of freedom, i.e., a first degree of freedom 11 in the X-axis direction, a second degree of freedom 12 in the Y-axis direction, a third degree of freedom 13 in the Z-axis direction, a fourth degree of freedom 14 about the X axis, a fifth degree of freedom 15 about the Y axis, and a sixth degree of freedom 16 about the Z axis. Owing to the six axial degrees of freedom, for example, the position adjuster 10 can move the syringe 50 or the needle of the syringe 50 (hereinafter referred to as a syringe needle) to a desired position and in a desired direction in accordance with control by the information processing device 100.

The fourth degree of freedom 14 about the X axis among the six axial degrees of freedom may be, for example, positioned when the syringe 50 is attached to the position adjuster 10 (or when the syringe needle is attached to the syringe 50). In this case, as long as the position adjuster 10 has five or more axial degrees of freedom, the position adjuster 10 can move the syringe 50 or the syringe needle to a desired position and in a desired direction. Thereby, even the blood vessel of the tail of a mouse thinner than the blood vessel of a human or the like can be punctured more appropriately. Also, the puncture becomes possible along a direction perpendicular to the cross-sectional surface of the tail in addition to a direction in which the tail extends. This can perform the puncture appropriately.

The first camera 20a and the second camera 20b are a camera configured to photograph (capture an image of) the tail 41 from different directions. Preferably, the first camera 20a and the second camera 20b are a visible light camera. The skin of the tail 41 of a mouse or the like is thinner than that of the human arm or the like. Thus, advantageously, the tail vein is readily detected even in the visible light region.

The retainer (retaining portion) 30 is used for keeping (retaining) a mouse or the like to be caused to undergo the tail vein injection so as not to move at the time of the tail vein injection. The animal to be caused to undergo the tail vein injection is not limited to a mouse, and may be any other animal, such as a rat, a guinea pig, a Mongolian gerbil, a hamster, a ferret, or the like. The following description will be made in relation to the case in which the animal to be caused to undergo the tail vein injection is a mouse as an example.

The fixture 40 is, for example, a fixture (jig) that includes a straight portion and a bent portion, and is configured to retain the tail 41. The syringe 50 is a syringe configured to puncture the tail vein. As used herein, the puncture refers to inserting the syringe needle into the blood vessel or the like from the exterior of the body for sampling of a biological fluid or tissue, or for infusion of a drug or the like.

The information processing device 100 has, for example, a configuration of a computer, and is configured to control the position adjuster 10 having five or more axes, the first camera 20a, the second camera 20b, and the like by executing one or more predetermined programs.

The configuration of the tail vein injection system 1 illustrated in FIG. 1 is just an example. The tail vein injection system 1 may be, for example, a tail vein injection device embedded in a single apparatus. Also, the tail vein injection system 1 may include three or more cameras configured to photograph the tail 41.

### <Outline of Tail Vein Injection Method>

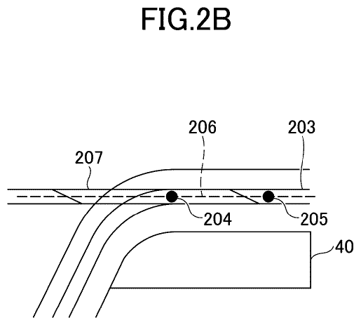

FIGS. 2A and 2B are diagrams illustrating an image of a tail vein injection method according to an embodiment of the present disclosure. As illustrated in FIG. 2A, the fixture 40 includes: a straight portion 201 in which a part of the tail 41 is retained as a straight part in a straight line; and a bent portion 202 in which another part of the tail 41 is retained such that the curvature of the another part is higher than that of the straight part.

The information processing device 100 uses cameras (the first camera 20a, the second camera 20b, and the like) to photograph the tail 41 retained by the fixture 40, and calculates the three-dimensional positions of two points on a tail vein 203 based on the photographed images. For example, the information processing device 100 calculates the three-dimensional positions of a point 204 serving as a puncture point and a point 205 serving as a puncture end point in the straight part of the tail 41 retained by the straight portion 201. Also, the information processing device 100 uses the position adjuster 10 having five or more axes, thereby aligning the position of a syringe needle 207 on a line 206 passing through the points 204 and 205.

Thus, according to the tail vein injection system 1 according to the present embodiment, as illustrated in FIG. 2B, the syringe needle 207 is inserted along the line 206 passing through the points 204 and 205. For example, according to the tail vein injection system 1, the puncture is performed using the point 204 as a puncture point and the point 205 as a puncture end point.

According to the existing techniques, the puncture is performed in a direction oblique to the blood vessel. Thus, when further advancing the syringe needle 207 after the syringe needle 207 reaches the blood vessel, the syringe needle may penetrate the blood vessel. Meanwhile, according to the tail vein injection system 1 according to the present embodiment, as illustrated in FIG. 2B, the syringe needle 207 can be further advanced after the syringe needle 207 reaches the tail vein 203. This facilitates the puncture of the tail vein of a mouse or the like that is thinner than the blood vessel of a human.

The puncture of injecting the syringe needle 207 into the tail vein 203 may be performed by the tail vein injection system 1 using an actuator or the like, or may be performed by a user.

### <Configurations of Fixture and Retainer>

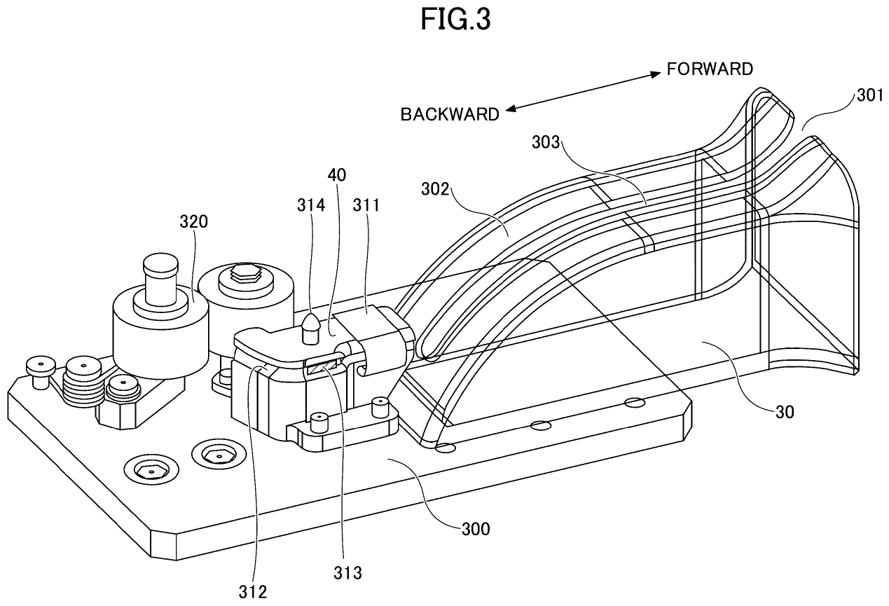

FIG. 3 is a diagram illustrating configuration examples of the fixture and the retainer according to an embodiment of the present disclosure. In the example of FIG. 3, the fixture 40, the retainer 30, a clamp 320, and the like are attached to a base 300 serving as a base portion.

The retainer (retaining portion) 30 has a cylindrical shape that is open forward of a mouse. The mouse is put into the retainer 30 through an opening 301 with the head of the mouse facing forward. An upper wall 302 of the retainer 30 is provided with a slit 303 for passing the tail 41 of the mouse, and the tail 41 of the mouse can be readily set in the fixture 40.

The upper wall 302 of the retainer 30 is inclined so as to gradually decrease in height from forward to backward of the retainer 30. Thereby, the retainer 30 can retain the buttocks of the mouse while preventing the back feet of the mouse from kicking the wall. Further, by providing the opening 301 forward of the retainer 30, an effect of the tail 41 of the mouse extending further firmly can be obtained because the mouse tries to escape from the opening 301.

The fixture 40 includes a tourniquet 311. This tourniquet 311 can fix the tail 41 and dilate the tail vein.

Also, the fixture 40 includes a groove 312 that receives the tail. The groove 312 of the straight portion 201 is provided with a hole 313 for suction of the tail 41 and for irradiation with light. For example, by connecting a suction pump to a suction port 314 and driving the suction pump, a part of the tail 41 is suctioned into the hole 313 provided in the straight portion 201 of the fixture 40, and as a result the tail 41 is retained in a straight line. Also, by turning on a light source provided in the fixture 40, light is irradiated from the hole 313. This facilitates detection of the tail vein from an image captured by the first camera 201a or the second camera 201b.

### <Configuration of Clamp>

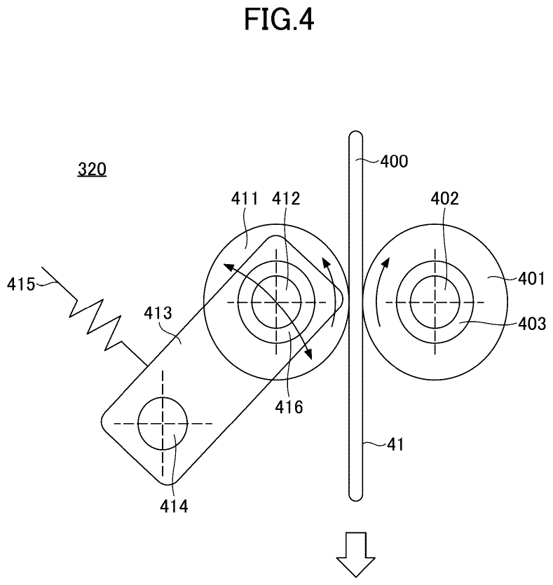

The clamp 320 has, for example, a configuration as illustrated in FIG. 4. FIG. 4 is a diagram illustrating a configuration example of a clamp according to an embodiment of the present disclosure. In the example of FIG. 4, the clamp 320 includes two rollers 401 and 411 configured to retain a tail tip 400 of the tail 41. The roller 401 is attached to a shaft 402 fixed to the base 300. The roller 401 includes a one-way clutch 403 configured to transmit a rotational force only in one direction.

The roller 411 is attached via a shaft 412 to a movable arm 413 that is attached via a shaft 414 to the base 300. Similar to the roller 401, the roller 411 includes a one-way clutch 416. The roller 411 can retain and release the tail tip 400 by moving the movable arm 413 by utilizing the elasticity of a spring 415 attached to the movable arm 413. As described above, the clamp 320 is configured such that a retention force to retain the tail tip 400 is increased when the tail 41 of the mouse is pulled by the mouse in an arrow direction.

### <Functional Configuration of Information Processing Device>

The information processing device 100 has a configuration of a computer, as illustrated in FIG. 5. The information processing device 100 is configured to execute one or more predetermined programs by the computer, thereby implementing an image obtaining unit 501, a detecting unit 502, a calibrating unit 503, a storage unit 504, a calculating unit 505, an adjusting unit 506, a puncturing unit 507, and the like. At least some of the above functional components may be implemented by hardware.

The image obtaining unit 501 is configured, for example, to control the first camera 20a and the second camera 20b to photograph a subject, and obtain a photographed image. For example, the image obtaining unit 501 obtains: a first image obtained by photographing the straight part of the tail 41 by the first camera 20a; and a second image obtained by photographing the straight part of the tail 41 by the second camera 20b. In the following description, any camera among the first camera 20a and the second camera 20b will be referred to as “camera 20”. Preferably, the camera 20 is a visible light camera.

The detecting unit 502 is configured to detect the tail vein 203, the syringe needle 207, and the like from the image obtained by the image obtaining unit 501. Preferably, the detecting unit 502 detects the tail vein 203, the syringe needle 207, and the like in the visible light region.

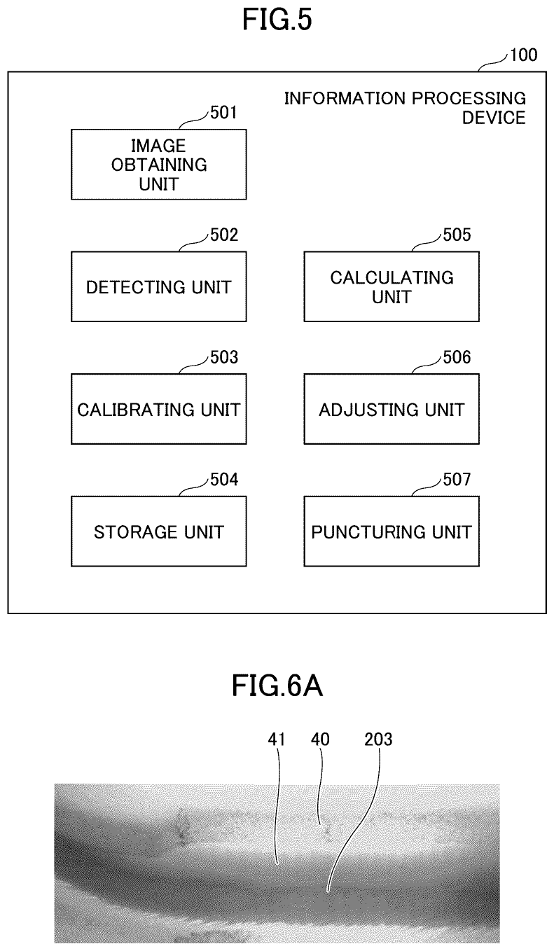

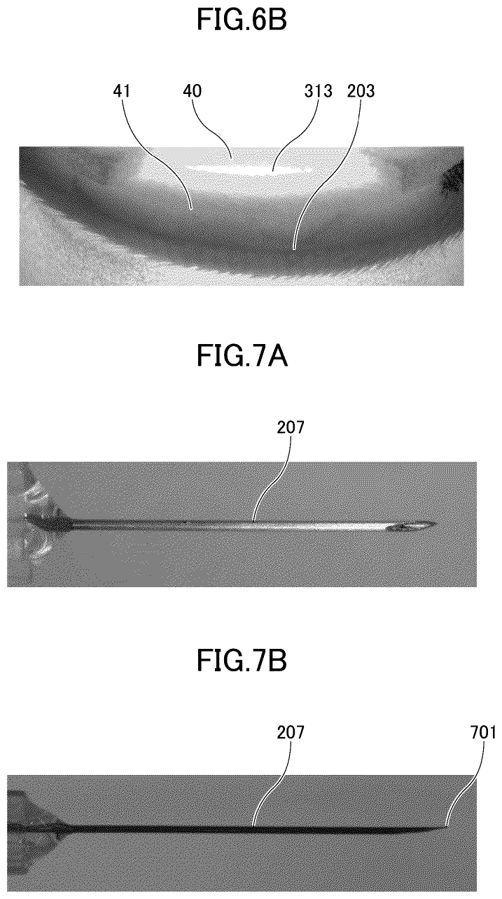

FIG. 6A illustrates an example of an image of the tail 41 photographed by the camera 20, with the tail 41 being retained by the fixture 40. Because the fixture 40 according to the present embodiment includes the hole 313 for suction of the tail 41 and for irradiation with light, the tail 41 can be retained in a straight line through suction as illustrated in FIG. 6A. Also, the tail 41 of a mouse is much thinner than the human arm or the like, and thus the tail vein 203 can be detected from the image captured by the visible light camera by use of visible light irradiated through the hole 313 of the fixture 40.

For example, the detecting unit 502 determines the darkest point as a candidate of the tail vein 203 from the image captured by the visible light camera, and performs linear fitting on the candidate to detect the tail vein 203. This process is represented by the following Expression (1), where the tail vein 203 is represented by ax+b in the image.

$$\begin{matrix}[ Math . 1 ] & \\ {arg min}_{a , b} {\sum }_{x} {( ( ax + b ) - {arg min}_{y} I ( x , y ) )}^{2} & ( 1 )\end{matrix}$$

In Expression (1), I(x, y) represents illuminance of a pixel in a pixel (x, y). When the image is represented in RGB colors, the tail vein 203 is represented as a dark region in the R (Red) channel and the G (Green) channel. Therefore, the detecting unit 502 may perform linear fitting for the R channel and the G channel separately, and employ the detection result of the channel with less outliers.

FIG. 6B illustrates an example of an image of the tail 41 photographed by the camera 20, with no suction being performed in the fixture 40. It is found that the tail 41 of the mouse is bent as illustrated in FIG. 6B when no suction is performed. Therefore, when no suction is performed, it is challenging to retain the tail 41 of the mouse in a straight line. Also, in this case, light leaks out of the hole 313, and thus the tail vein 203 is blurred and detection of the tail vein 203 is also challenging.

Meanwhile, the fixture 40 according to the present embodiment includes the hole 313 for suction of the tail 41 and for irradiation with light. Thus, the tail 41 is readily retained in a straight line, and the tail vein is readily detected using a visible light camera.

FIG. 7A illustrates an example of an image of the syringe needle 207 photographed by the camera 20. As illustrated in FIG. 7A, the needle tip of the syringe needle 207 is not readily detected from this image due to specular reflection of the metal surface. In such a case, as illustrated in FIG. 7B, it is effective to backlight the syringe needle 207, and detect the syringe needle 207 as a silhouette. However, it may be undesirable in terms of space and cost to provide each of the first camera 20a and the second camera 20b with a backlight configured to backlight the syringe needle 207 at sufficient illuminance.

Therefore, in the present embodiment, a back panel serving as the background of the syringe needle 207 is covered by a phosphor, and ultraviolet rays are emitted from the camera 20 side, thereby realizing a thin back panel with uniform illuminance. The ultraviolet rays emitted from the camera 20 side do not affect the image captured by the camera 20, and the back panel covered by the phosphor can emit light in the visible light region. Thus, the detecting unit 502 can readily detect a needle tip 701 of the syringe needle 207 from, for example, the image as illustrated in FIG. 7B.

When the camera 20 is an infrared camera as in the existing techniques, it may be challenging to realize the same configuration at the same cost. This is because phosphors emitting light in the infrared region are not common.

The description of the functional configuration of the information processing device 100 will be continued with reference to FIG. 5. The calibrating unit 503 is configured to perform a first calibration process (overall calibration) of calibrating the camera parameters of the camera 20, and two or more positions from among the position of the fixture 40, the position of the position adjuster 10, and the position of the needle tip 701 of the syringe needle 207. When the syringe 50 or the syringe needle 207 is replaced, the calibrating unit 503 performs a second calibration process (needle tip calibration) through which re-calibration is possible by calibrating a smaller number of members than that in the first calibration process, including the position of the needle tip 701 of the syringe needle 207.

The following will describe an example in which the calibrating unit 503 calibrates the camera parameters of the camera 20, the position of the position adjuster 10, and the position of the needle tip 701 of the syringe needle 207.

In order to correctly puncture the mouse tail vein 203 having a diameter of about 0.3 millimeters (mm) with the syringe needle 207, it is desirable to correctly calibrate the position of the needle tip 701 of the syringe needle 207 with respect to the position adjuster 10, and system parameters, such as camera parameters and the like. In the present embodiment, this calibration process is called a first calibration process. However, it is not desirable in terms of time to perform the first calibration process every time the puncture is performed. This is because the syringe 50 or the syringe needle 207 is always replaced after the puncture.

Therefore, the calibrating unit 503 according to the present embodiment is configured to perform re-calibration by calibrating the position of the needle tip 701 of the syringe needle 207 with respect to the position adjuster 10 when the syringe 50 or the syringe needle 207 is replaced. In the present embodiment, this calibration process is called a second calibration process.

In the first calibration process, the calibrating unit 503 controls the position adjuster 10 to gradually move the position of the needle tip 701 of the syringe needle 207 and photograph the needle tip 701 by the camera 20, and solves the optimization problem of Expression (2), thereby calibrating the entire system.

$$\begin{matrix}[ Math . 2 ] & \\ {arg min}_{q , {c}_{1} , {c}_{2}} {\sum }_{i = 1}^{n} {\sum }_{j = 1 , 2} {‖ {p}_{i}^{j} - f ( {H}_{i} q \vert {c}_{j} ) ‖}^{2} & ( 2 )\end{matrix}$$

In Expression (2), c is camera parameters of the camera 20, and represents, for example, the position or the angle of view (e.g., parameters of a lens) of the first camera 20a or the second camera 20b. H represents the position of the position adjuster 10 (e.g., the position of the origin). q represents where the needle tip 701 of the syringe needle 207 is located as seen from the position of the position adjuster 10. A function f is a function that converts a point on the three-dimensional space into a pixel on an image of the camera 20 using the camera parameters c. p represents the position of the needle tip 701 on the image of the camera 20.

The calibrating unit 503 performs a calibration process of the entire system (first calibration process) by solving the optimization problem of Expression (2) so as to minimize the difference between the position of the needle tip 701 on the image of the camera 20 represented by p, and the position of the needle tip 701 converted by the function f( ).

In the second calibration process, the calibrating unit 503 calibrates only q, which represents the position of the needle tip 701 with respect to the position adjuster 10. This second calibration process can be performed, for example, only by setting the position adjuster 10 to the home position and photographing one set of images using the first camera 20a and the second camera 20b. Thus, the processing time can be significantly reduced compared to the first calibration process.

The storage unit 504 is configured to store, for example, the camera parameters c of the first camera 20a and the second camera 20b, the position H of the position adjuster 10, the position q of the needle tip 701 with respect to the position adjuster 10, and the like, which are obtained by the calibrating unit 503.

The calculating unit 505 is configured to perform a calculation process of calculating the three-dimensional positions of two points on the tail vein 203 based on images of the tail 41 captured by the camera 20. The calculating unit 505 calculates the three-dimensional positions of the two points on the tail vein based on images of the tail captured under conditions in which either or both of the photographing position and the photographing direction are different. For example, the calculating unit 505 uses the first image captured by the first camera 20a and the second image captured by the second camera 20b, thereby calculating the three-dimensional positions of the points 204 and 205 described in FIG. 2A. Details of the calculation process performed by the calculating unit 505 will be described below with reference to flowcharts and drawings.

The adjusting unit 506 is configured to perform an adjustment process of adjusting the position and the posture of the syringe needle by using the position adjuster having five or more axes. For example, as described in FIG. 2A, the adjusting unit 506 uses the position adjuster having five or more axes, thereby aligning the position of the syringe needle 207 on the line 206 passing through the points 204 and 205 obtained by the calculating unit 505. The adjusting unit 506 can accurately determine the position of the needle tip 701 of the syringe needle 207 by using the first image captured by the first camera 20a and the second image captured by the second camera 20b based on the calibration result obtained by the calibrating unit 503. Therefore, the adjusting unit 506 can align the position of the syringe needle 207 on the line 206 passing through the points 204 and 205 by controlling the position adjuster 10 to gradually change the posture of the syringe 50 while obtaining the first image and the second image and confirming the position of the needle tip 701.

As illustrated in FIG. 2B, the puncturing unit 507 is configured to insert the syringe needle 207 along the line 206 passing through the points 204 and 205. For example, the puncturing unit 507 performs the puncture using the point 204 as a puncture point and the point 205 as a puncture end point.

According to the tail vein injection system 1 according to the present embodiment, the puncturing unit 507 can have various configurations. For example, the tail vein injection system 1 may stop the process after aligning the position of the syringe needle 207 on the line 206 passing through the points 204 and 205, and the puncture process may be performed, for example, by a human hand or the like. In this case, the information processing device 100 may not include the puncturing unit 507.

Also, the puncturing unit 507 may detect, after the puncture, the pressure of a drug solution to be injected, and control the injection of the drug solution based on the detected pressure. For example, the puncturing unit 507 may stop the puncture when the pressure exceeds a threshold on the basis of the fact that the pressure is high if the syringe needle 207 is not correctly inserted into the tail vein 203. Alternatively, the puncturing unit 507 may determine, from an image, whether or not the syringe needle 207 is inserted into the tail vein 203 on the basis of the fact that when the syringe needle 207 is inserted into the tail vein 203, blood is extracted into the syringe 50 by pulling the piston of the syringe 50.

As described above, according to the tail vein injection system 1 according to the present embodiment, the puncturing unit 507 may have any configuration as long as the puncturing unit 507 performs the process of aligning the position of the syringe needle 207 on the line 206 passing through the points 204 and 205.

### <Flow of Process>

Next, a flow of a process of the tail vein injection method according to the present embodiment will be described.

### (Process of Tail Vein Injection System)

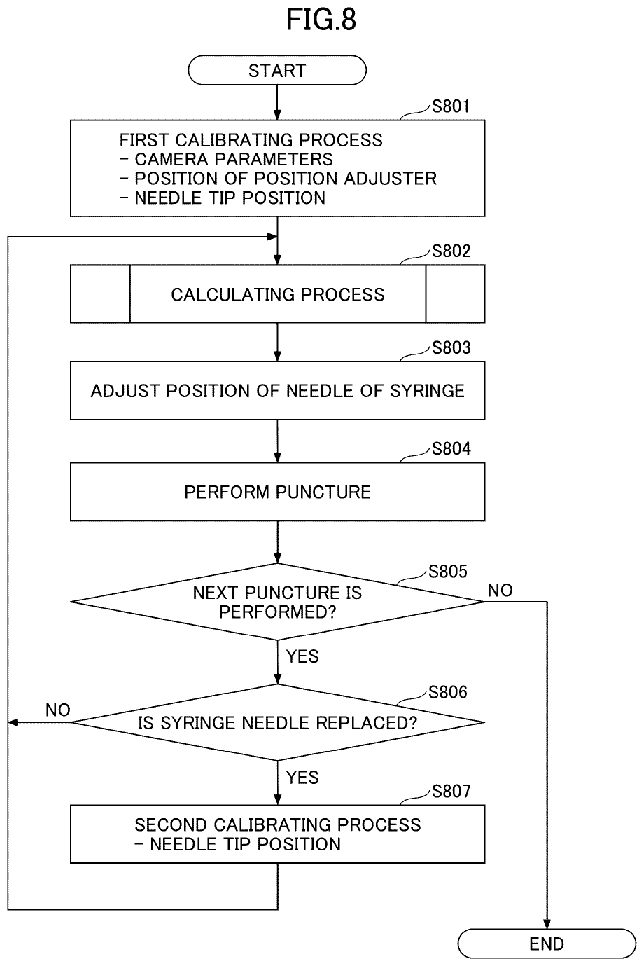

FIG. 8 is a flowchart illustrating a flow of a process of a tail vein injection system according to an embodiment of the present disclosure. This process is an example of the process performed by the information processing device 100 in the tail vein injection system 1 as illustrated in FIG. 1.

In step S801, the calibrating unit 503 of the information processing device 100 performs a first calibration process of calibrating, for example, the camera parameters of the first camera 20a and the second camera 20b, the position of the position adjuster 10, and the position of the needle tip 701 of the syringe needle 207. For example, as described above, the calibrating unit 503 controls the position adjuster 10 to gradually move the position of the needle tip 701 of the syringe needle 207, photographs the needle tip 701 by the camera 20, and solves the optimization problem of Expression (2), thereby calibrating the entire system.

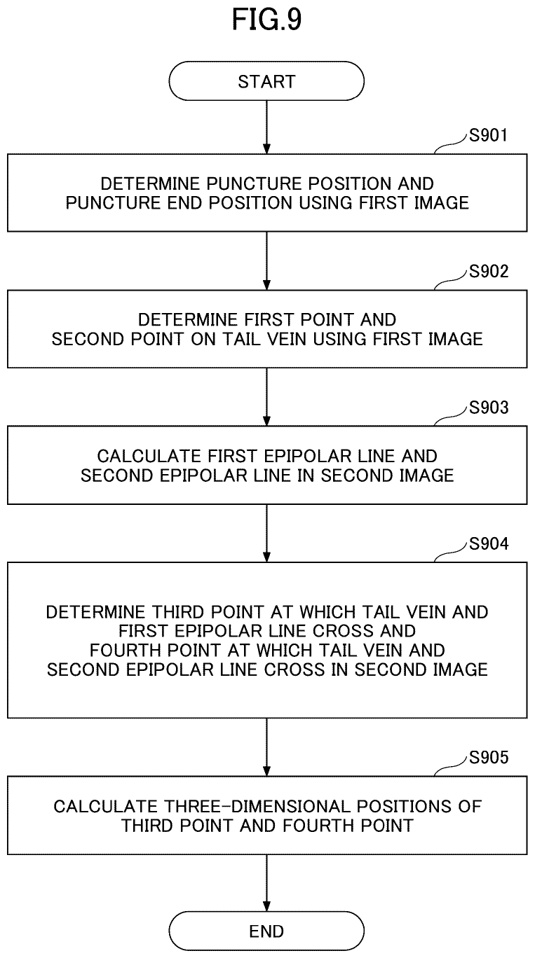

In step S802, the calculating unit 505 of the information processing device 100 performs a calculation process of calculating the three-dimensional positions of the points 204 and 205 by using the first image captured by the first camera 20a and the second image captured by the second camera 20b. Details of the calculation process will be described below with reference to FIGS. 9 to 14.

In step S803, the adjusting unit 506 of the information processing device 100 uses the position adjuster 10 having five or more axes as described with reference to FIG. 2A, thereby aligning the position of the syringe needle 207 on the line 206 passing through the points 204 and 205 obtained by the calculating unit 505.

In step S804, the puncturing unit 507 of the information processing device 100 inserts the syringe needle 207 into the tail vein 203 along the line 206 passing through the points 204 and 205 as illustrated in FIG. 2B. For example, the puncturing unit 507 performs the puncture using the point 204 as a puncture point and the point 205 as a puncture end point.

In step S805, the information processing device 100 determines whether or not to perform a next puncture. When a next puncture is performed, the information processing device 100 causes the process to proceed to step S806. Meanwhile, when a next puncture is not performed, the information processing device 100 ends the process of FIG. 8.

When the process proceeds to step S806, the information processing device 100 determines whether or not the syringe needle 207 (or the syringe 50) or the like has been replaced. When the syringe needle 207 or the like has been replaced, the information processing device 100 causes the process to proceed to step S807. Meanwhile, when the syringe needle 207 has not been replaced, the information processing device 100 returns the process to step S802.

As another example, when the syringe needle 207 has not been replaced, the information processing device 100 may display a message for requesting replacement of the syringe needle 207 and suspend the process until the syringe needle 207 is replaced. The information processing device 100 may determine whether or not the syringe needle 207 has been replaced based on an input or the like performed by a user or based on an image captured by the camera 20.

When the process proceeds to step S807, the calibrating unit 503 of the information processing device 100 performs a second calibration process of calibrating the position of the needle tip 701 of the syringe needle 207 with respect to the position adjuster 10, and returns the process to step S802 after the calibration.

Through the above process, the tail vein injection system 1 can facilitate the puncture of the tail vein as described with reference to FIGS. 2A and 2B. In addition, the tail vein injection system 1 can significantly reduce the time required for calibration even if the puncture is performed a plurality of times while replacing the syringe needle 207 or the syringe 50 every time the puncture is performed.

### (Calculation Process)

FIG. 9 is a flowchart illustrating an example of a calculation process according to an embodiment of the present disclosure. This process is an example of a calculation process performed by the calculating unit 505 of the information processing device 100 in step S802 of FIG. 8.

The following description will be made assuming that the camera parameters of the first camera 20a and the second camera 20b are known by the first calibration process or the second calibration process. The camera parameters include the position and the posture, the focal length, the optical center, the distortion correction parameter, or the like of the first camera 20a and the second camera 20b. It is also assumed that the positional relationship between the fixture 40 and the first and second cameras 20a and 20b is fixed.

In step S901, the calculating unit 505 determines the puncture position and the puncture end position using the first image captured by the first camera 20a.

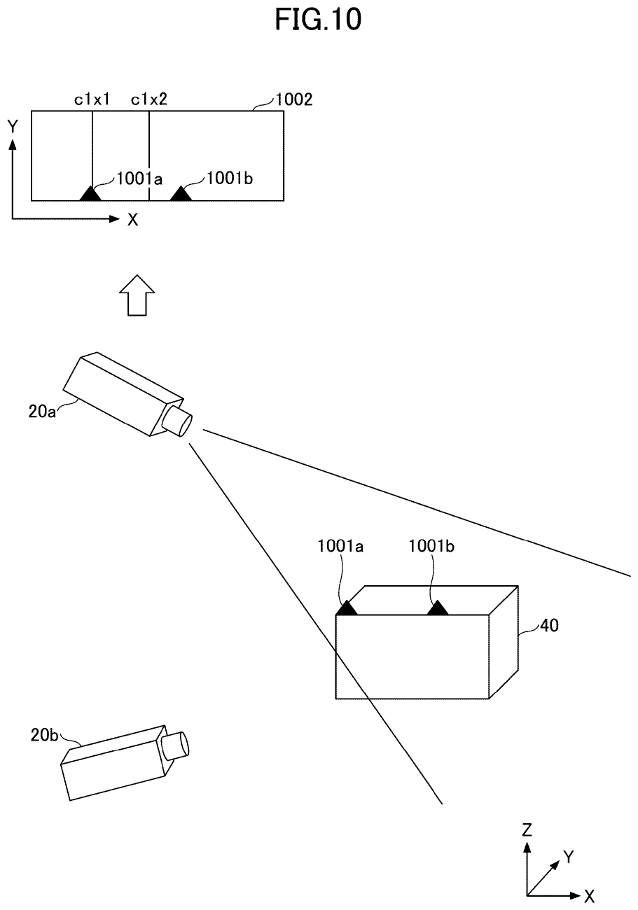

As a preferable example, as illustrated in FIG. 10, the fixture 40 includes a first marking 1001a and a second marking 1001b for positioning. By photographing this fixture 40 by the first camera 20a, an image 1002 obtained by photographing the first marking 1001a and the second marking 1001b on the fixture 40 is obtained as illustrated in FIG. 10.

For example, the calculating unit 505 determines the position of the first marking 1001a in the X-axis direction as a puncture position c1x1 in the image 1002. Also, the calculating unit 505 determines a puncture end position c1x2 in the X-axis direction based on the distance to the puncture end point. For example, when the distance between the first marking 1001a and the second marking 1001b is D and the distance to the puncture end point is set to d, the calculating unit 505 determines, as the puncture end position c1x2, a position d/D in the X-axis direction from the puncture position c1x1.

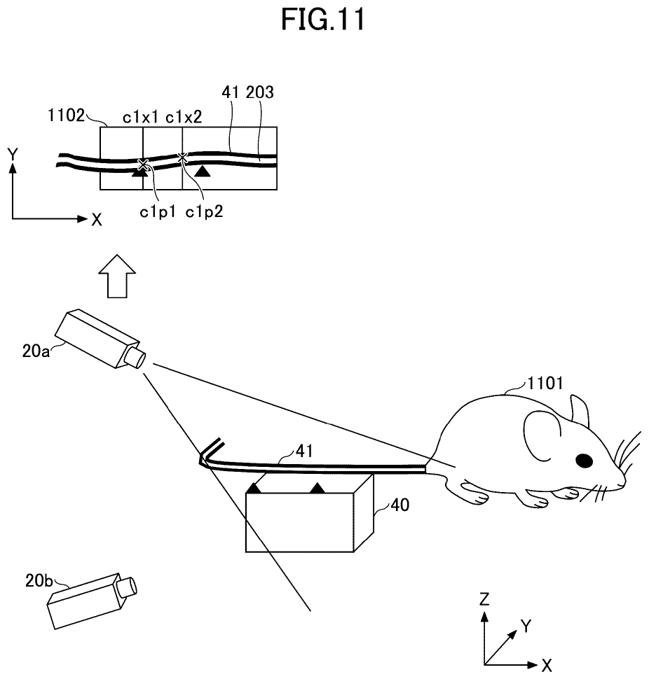

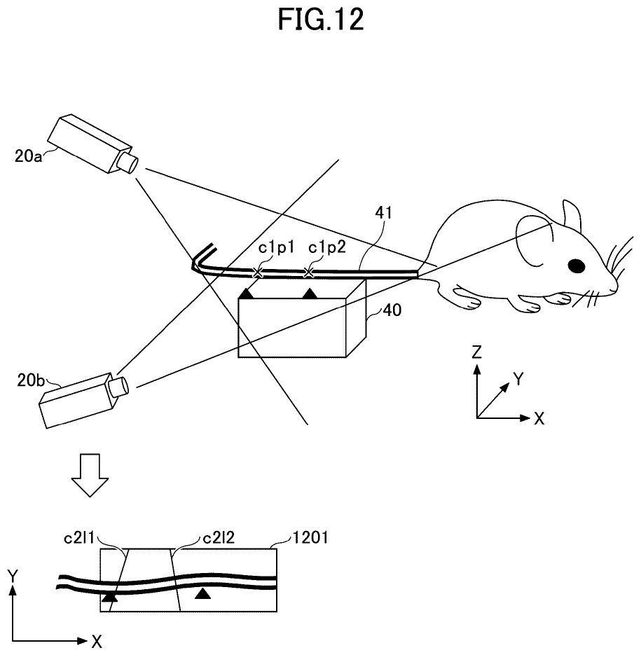

In step S902, the calculating unit 505 determines a first point and a second point on the tail vein using the first image captured by the first camera 20a. For example, as illustrated in FIG. 11, the calculating unit 505 obtains a first image 1102 by photographing the tail 41 by the first camera 20a using the image obtaining unit 501 in a state in which the tail 41 of a mouse 1101 is fixed to the fixture 40. Also, the calculating unit 505 detects the tail vein 203 from the first image 1102 using the detecting unit 502. Further, in the first image 1102, the calculating unit 505 sets, as a first point c1p1, a position at which the puncture position c1x1 in the X-axis direction and the tail vein 203 cross each other, and sets, as a second point c1p2, a position at which the puncture end position c1x2 in the X-axis direction and the tail vein 203 cross each other.

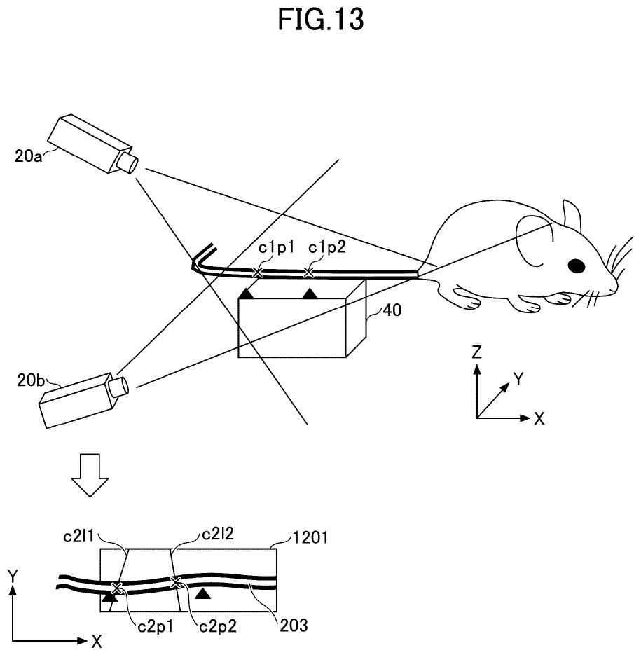

In step S903, the calculating unit 505 calculates a first epipolar line c2l1 and a second epipolar line c2l2 in a second image obtained by photographing the tail 41 by the second camera 20b. For example, as illustrated in FIG. 12, the calculating unit 505 uses the image obtaining unit 501 to obtain a second image 1201 obtained by photographing the tail 41 by the second camera 20b in a state in which the tail 41 of the mouse 1101 is fixed to the fixture 40. Also, in the second image 1201, the calculating unit 505 calculates the first epipolar line c2l1 connecting the first camera 20a and the first point c1p1.

A straight line connecting the first camera 20a and the first point c1p1 is projected as a straight line on an image from the second camera 20b. This straight line is referred to as an epipolar line. When the coordinates of the first point c1p1 are not known, the first point c1p1 captured by the first camera 20a is understood to be on the first epipolar line c2l1.

Similarly, the calculating unit 505 calculates a second epipolar line c2l2 connecting the first camera 20a and the second point c1p2 in the second image 1201.

In step S904, as illustrated in FIG. 13, the calculating unit 505 obtains, in the second image 1201, a third point c2p1 at which the tail vein 203 and the first epipolar line c2l1 cross each other, and a fourth point c2p2 at which the tail vein 203 and the second epipolar line c2l2 cross each other.

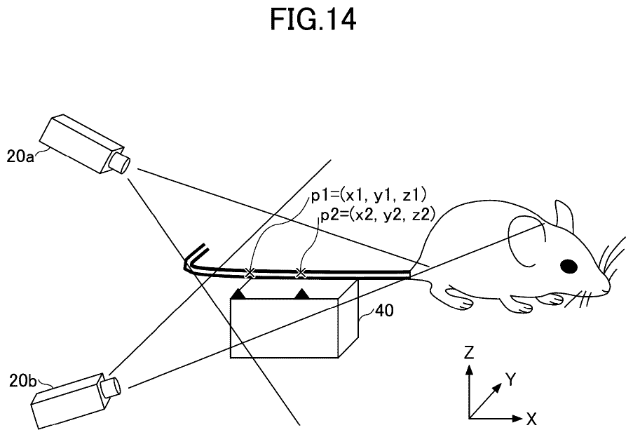

In step S905, the calculating unit 505 calculates the three-dimensional positions of the third point c2p1 and the fourth point c2p2. The first point c1p1 in the first image 1102 and the third point c2p1 in the second image 1201 indicate the same point (e.g., a point p1 in FIG. 14). Therefore, the calculating unit 505 can calculate the three-dimensional coordinates (x1, y1, z1) of the point p1 using the camera parameters of the first camera 20a and the second camera 20b, the first image 1102, and the second image 1201, for example, by a publicly known triangulation method or the like. This point p1 corresponds to the point 204 in FIG. 2A.

Similarly, the second point c1p2 in the first image 1102 and the fourth point c2p2 in the second image 1201 indicate the same point (e.g., a point p2 in FIG. 14). Therefore, the calculating unit 505 can calculate the three-dimensional coordinates (x2, y2, z2) of the point p2 using the camera parameters of the first camera 20a and the second camera 20b, the first image 1102, and the second image 1201, for example, by a publicly known triangulation method or the like. This point p2 corresponds to the point 205 in FIG. 2A.

Through the above process, the information processing device 100 can calculate the three-dimensional positions of two points on the tail vein 203 (e.g., the points 204 and 205 in FIG. 2A).

According to an embodiment of the present disclosure, it is possible to provide a tail vein injection system that facilitates a puncture of the tail vein.

### [Configuration of Information Processing Device]

A part or all of the information processing device 100 according to the above-described embodiment may be configured by hardware, or by information processing of software (program) executed by a CPU (Central Processing Unit), a GPU (Graphics Processing Unit), or the like. When the information processing device 100 is configured by the information processing of the software, the information processing of the software may be executed by storing the software, which realizes at least a part of the functions of the information processing device 100 in the above-described embodiment, in a non-transitory storage medium (a non-transitory computer-readable medium, e.g., a CD-ROM (Compact Disc-Read Only Memory), a USB (Universal Serial Bus) memory, or the like) and by reading the non-transitory storage medium in a computer. Also, the software may be downloaded via a communication network. Further, the information processing by software may be executed by hardware with a part or all of the software being mounted in circuits such as an ASIC (Application Specific Integrated Circuit), a FPGA (Field Programmable Gate Array), or the like.

The storage medium storing the software may be a removable storage medium, such as an optical disk or the like, and may be a fixed storage medium, such as a hard disk, a memory, or the like. Also, the storage medium may be provided internally of the computer (a main storage device, an auxiliary storage device, or the like), or provided externally to the computer.

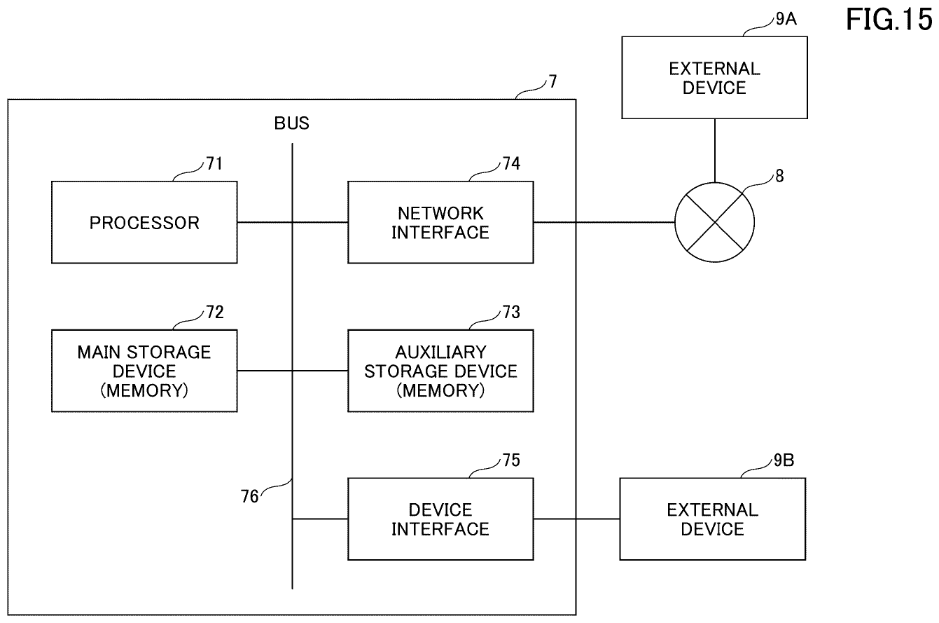

FIG. 15 is a block diagram illustrating a hardware configuration of the information processing device 100 in the above-described embodiment. As one example, the information processing device 100 includes a processor 71, a main storage device 72 (memory), an auxiliary storage device 73 (memory), a network interface 74, and a device interface 75. These are connected to each other via a bus 76 and may be realized as a computer 7.

The computer 7 of FIG. 15 includes one of each of the constituting elements, but may include multiple constituting elements that are the same. Also, FIG. 15 illustrates one computer 7, but the software may be installed in multiple computers, which may each execute the same or different partial processes of the software. In this case, the computers may be in the form of distributed computing in which the computers communicate with each other via the network interface 74 or the like, thereby executing the process. That is, the information processing device 100 in the above-described embodiment may be configured as a system in which one or more computers execute instructions stored in one or more storage devices, thereby realizing the functions. Also, the information processing device 100 in the above-described embodiment may be configured such that information transmitted from a terminal is processed by one or more computers provided on the cloud and the result of the processing is transmitted to the terminal.

Various computations of the information processing device 100 in the above-described embodiment may be executed in parallel using one or more processors or multiple computers connected via a network. Also, various computations may be distributed among multiple computation cores inside the processor, and executed through parallel processing. Also, a part or all of the processing, means, or the like of the present disclosure may be realized by at least one of a processor and a storage device provided on the cloud communicable with the computer 7 via a network. In this way, the devices in the above-described embodiment may be in the form of parallel computing by one or more computers.

The processor 71 may be an electronic circuit (e.g., a process circuit, a processing circuit, a processing circuitry, a CPU, a GPU, a FPGA, an ASIC, or the like) configured to perform control of a computer, an arithmetic process, or both. Also, the processor 71 may be a general-purpose processor or may be a semiconductor device including a dedicated processing circuit designed to perform specific calculation or both of the general-purpose processor and the dedicated processing circuit. Further, the processor 71 may include an optical circuit or computing functions based on quantum computing.

The processor 71 may perform computation processing based on data and software input from the devices provided internally of the computer 7, and output a computation result and a control signal to the devices. The processor 71 may control the constituting elements of the computer 7 by executing an OS (Operating System), an application, or the like of the computer 7.

The information processing device 100 in the above-described embodiment may be realized by one or more processors 71. Here, the processor 71 may refer to one or more electronic circuits disposed on a single chip, or may refer to one or more electronic circuits disposed on two or more chips or on two or more devices. When using two or more electronic circuits, each of the electronic circuits may communicate by wire or wirelessly.

The main storage device 72 may store instructions to be executed by the processor 71, various data, and the like, and the information stored in the main storage device 72 may be read out by the processor 71. The auxiliary storage device 73 is a storage device other than the main storage device 72. These storage devices refer to given electronic components capable of storing electronic information, and may be semiconductor memories. The semiconductor memory may be a volatile memory or a non-volatile memory. The storage device for storing the various data and the like in the information processing device 100 in the above-described embodiment may be realized by the main storage device 72 or the auxiliary storage device 73, or may be realized by an internal memory provided internally of the processor 71. For example, the storage unit 504 in the above-described embodiment may be realized by the main storage device 72 or the auxiliary storage device 73.

When the information processing device 100 in the above-described embodiment is configured with at least one storage device (memory) and at least one processor connected (coupled) to the at least one storage device, at least one processor may be connected to a single storage device. Alternatively, at least one storage device may be connected to a single processor. Also, at least one processor of the multiple processors may be connected to the at least one storage device of the multiple storage devices. Also, such a configuration may be realized by a storage device and a processor included in multiple computers. Further, the configuration may include the storage device integrated with the processor (e.g., a cache memory including a L1 cache, a L2 cache, or the like).

The network interface 74 is an interface for connecting to a communication network 8, by wire or wirelessly. The network interface 74 may use an appropriate interface such as an interface conforming to existing communication standards. Exchange of information with an external device 9A connected via the communication network 8 may be performed via the network interface 74. Note that, the communication network 8 may be a WAN (Wide Area Network), a LAN (Local Area Network), a PAN (Personal Area Network), or the like, or may be a combination thereof, as long as exchange of information is performed between the computer 7 and the external device 9A. Examples of the WAN include the Internet and the like. Examples of the LAN include the IEEE 802.11, ETHERNET (registered trademark), and the like. Examples of the PAN include Bluetooth (registered trademark), NFC (Near Field Communication), and the like.

The device interface 75 may be an interface, such as a USB or the like, that directly connects to an external device 9B.

The external device 9A is a device that is connected to the computer 7 via a network. The external device 9B is a device that is directly connected to the computer 7.

The external device 9A or the external device 9B may be an input device, as one example. The input device may be a device, such as a camera, a microphone, a motion capture device, various sensors, a keyboard, a mouse, a touch panel, or the like, and provides obtained information to the computer 7. Also, the external device 9A or the external device 9B may be a device including an input part, a memory, and a processor, such as a personal computer, a tablet terminal, a smartphone, or the like.

Also, the external device 9A or the external device 9B may be an output device, as one example. The output device may be a display device, such as a LCD (Liquid Crystal Display), an organic EL (Electro Luminescence) panel, or the like, or may be a speaker or the like that outputs voice or the like. Also, the external device 9A or the external device 9B may be a device including an output part, a memory, and a processor, such as a personal computer, a tablet terminal, a smartphone, or the like.

Also, the external device 9A or the external device 9B may be a storage device (memory). For example, the external device 9A may be a network storage or the like, and the external device 9B may be a storage, such as a HDD or the like.

Also, the external device 9A or the external device 9B may be a device having the functions of a part of the constituting elements of the information processing device 100 in the above-described embodiment. That is, the computer 7 may transmit a part or all of the processing results of the external device 9A or the external device 9B, or may receive a part or all of the processing results of the external device 9A or the external device 9B.

As described above, according to an embodiment of the present embodiment, it is possible to provide a tail vein injection system 1 that facilitates a puncture of the tail vein.

### NOTES

In the present specification (including the claims), if the expression “at least one of a, b, and c” or “at least one of a, b, or c” is used (including similar expressions), any one of a, b, c, a-b, a-c, b-c, or a-b-c is included. Multiple instances may also be included in any of the elements, such as a-a, a-b-b, and a-a-b-b-c-c. Further, the addition of another element other than the listed elements (i.e., a, b, and c), such as adding d as a-b-c-d, is included.

In the present specification (including the claims), in a case where an expression, such as “data as an input”, “using data”, “based on data”, “according to data”, or “in accordance with data” (including similar expressions) is used, such a case may, unless otherwise noted, encompass a case in which data themselves are used and a case in which data obtained by processing data (e.g., data obtained by adding noise, normalized data, feature extracted from data, and intermediate representation of data) are used. If it is described that any result can be obtained “based on data as an input”, “using data”, “based on data”, “according to data”, or “in accordance with data” (including similar expressions), unless otherwise noted, a case in which the result is obtained based on only the data is included, and a case in which the result is obtained affected by another data other than the data, factors, conditions, and/or states is included. If it is described that “data are output” (including similar expressions), unless otherwise noted, a case in which data themselves are used as an output is included, and a case in which data obtained by processing data in some way (e.g., data obtained by adding noise, normalized data, feature extracted from data, and intermediate representation of various data) are used as an output is included.

In the present specification (including the claims), if the terms “connected” and “coupled” are used, the terms are intended as non-limiting terms that include any of direct, indirect, electrically, communicatively, operatively, and physically connected/coupled. Such terms should be interpreted according to a context in which the terms are used, but a connected/coupled form that is not intentionally or naturally excluded should be interpreted as being included in the terms without being limited.

In the present specification (including the claims), if the expression “A configured to B” is used, a case in which a physical structure of the element A has a configuration that can perform the operation B, and a permanent or temporary setting/configuration of the element A is configured/set to actually perform the operation B may be included. For example, if the element A is a general-purpose processor, the processor may have a hardware configuration that can perform the operation B and be configured to actually perform the operation B by setting a permanent or temporary program (i.e., an instruction). If the element A is a dedicated processor or a dedicated arithmetic circuit, a circuit structure or the like of the processor may be implemented so as to actually perform the operation B irrespective of whether the control instruction and the data are actually attached.

In the present specification (including the claims), if a term indicating containing or possessing (e.g., “comprising/including” and “having”) is used, the term is intended as an open-ended term, including an inclusion or possession of an object other than a target object indicated by the object of the term. If the object of the term indicating an inclusion or possession is an expression that does not specify a quantity or that suggests a singular number (i.e., an expression using “a” or “an” as an article), the expression should be interpreted as being not limited to a specified number.

In the present specification (including the claims), even if an expression such as “one or more” or “at least one” is used in a certain description, and an expression that does not specify a quantity or that suggests a singular number (i.e., an expression using “a” or “an” as an article) is used in another description, it is not intended that the latter expression indicates “one”. Generally, an expression that does not specify a quantity or that suggests a singular number (i.e., an expression using “a” or “an” as an article) should be interpreted as being not necessarily limited to a particular number.

In the present specification, if it is described that a particular advantage/result is obtained in a particular configuration included in an embodiment, unless there is a particular reason, it should be understood that that the advantage/result may be obtained in another embodiment or other embodiments including the configuration. It should be understood, however, that the presence or absence of the advantage/result generally depends on various factors, conditions, states, and/or the like, and that the advantage/result is not necessarily obtained by the configuration. The advantage/result is merely an advantage/result that results from the configuration described in the embodiment when various factors, conditions, and/or states are satisfied, and is not necessarily obtained in the claimed invention that defines the configuration or a similar configuration.

In the present specification (including the claims), if a term such as “maximize”/“maximization” is used, it should be interpreted as appropriate according to a context in which the term is used, including obtaining a global maximum value, obtaining an approximate global maximum value, obtaining a local maximum value, and obtaining an approximate local maximum value. It also includes determining approximate values of these maximum values, stochastically or heuristically. Similarly, if a term such as “minimize”/“minimization” is used, they should be interpreted as appropriate, according to a context in which the term is used, including obtaining a global minimum value, obtaining an approximate global minimum value, obtaining a local minimum value, and obtaining an approximate local minimum value. It also includes determining approximate values of these minimum values, stochastically or heuristically. Similarly, if a term such as “optimize”/“optimization” is used, the term should be interpreted as appropriate according to a context in which the term is used, including obtaining a global optimum value, obtaining an approximate global optimum value, obtaining a local optimum value, and obtaining an approximate local optimum value. It also includes determining approximate values of these optimum values, stochastically or heuristically.

In the present specification (including the claims), if multiple hardware performs predetermined processes, each of the hardware may cooperate to perform the predetermined processes, or some of the hardware may perform all of the predetermined processes. Additionally, some of the hardware may perform some of the predetermined processes while other hardware may perform the remainder of the predetermined processes. In the present specification (including the claims), if an expression such as “one or more hardware perform a first process and the one or more hardware perform a second process” (including similar expressions) is used, the hardware that performs the first process may be the same as or different from the hardware that performs the second process. That is, the hardware that performs the first process and the hardware that performs the second process may be included in the one or more hardware. The hardware may include an electronic circuit, a device including an electronic circuit, or the like.

In the present specification (including the claims), if multiple storage devices (memories) store data, each of the multiple storage devices may store only a portion of the data or may store an entirety of the data. Also, a configuration in which some of the multiple storage devices store data may be included.

Although the embodiments of the present disclosure have been described in detail above, the present disclosure is not limited to the individual embodiments described above. Various additions, modifications, substitutions, partial deletions, and the like may be made without departing from the conceptual idea and spirit of the invention derived from the contents defined in the claims and the equivalents thereof. For example, in the embodiments described above, when numerical values or mathematical formulae are used for description, these are used for illustrative purposes and do not limit the scope of the present disclosure. Additionally, the orders of operations described in the embodiments are illustrative and do not limit the scope of the present disclosure.

What is claimed is:

1. A tail vein injection system, comprising:
at least one memory; and
at least one processor connected to the at least one memory, the at least one processor being configured to:
calculate information including a position of a tail vein based on one or more images of a tail;
adjust a position and a posture of a syringe needle based on the calculated information; and
after the adjusting, perform a puncture of the tail vein using the syringe needle.

2. The tail vein injection system according to claim 1, wherein
the at least one processor is configured to calibrate a position of the syringe needle.

3. The tail vein injection system according to claim 2, wherein
the at least one processor is configured to
upon replacement of the syringe needle, calibrate the position of the syringe needle after the replacement.

4. The tail vein injection system according to claim 3, wherein
the at least one processor is configured to
perform at least a first calibration and a second calibration, and
upon the replacement of the syringe needle, perform the second calibration without performing the first calibration,
the second calibration being calibrating a smaller number of members than in the first calibration.

5. The tail vein injection system according to claim 4, wherein
the first calibration is calibrating
two or more positions from among a position of a fixture configured to fix the tail, a position of a position adjuster, and the position of the syringe needle, and
a camera parameter of a camera configured to capture the one or more images.

6. The tail vein injection system according to claim 2, wherein
the at least one processor is configured to
calibrate a camera parameter.

7. The tail vein injection system according to claim 2, wherein
the at least one processor is configured to
calibrate the position of the syringe needle using an image obtained by photographing a needle tip of the syringe needle.

8. The tail vein injection system according to claim 1, further comprising:
a fixture configured to fix the tail,
the fixture including
a straight portion in which a shape of a part of the tail is retained in a straight line, and
a bent portion in which a shape of another part of the tail is retained such that a curvature thereof is higher than that of the straight line.

9. The tail vein injection system according to claim 8, wherein
the at least one processor is configured to
start the puncture of the another part of the tail that is bent at the bent portion, and
advance the syringe needle along a line passing through two points in the part of the tail.

10. The tail vein injection system according to claim 8, wherein
the straight portion of the fixture is closer to a root of the tail, and the bent portion of the fixture is closer to a tip of the tail.

11. The tail vein injection system according to claim 8, wherein
the fixture is configured to retain the part of the tail in the straight line through suction.

12. The tail vein injection system according to claim 8, wherein
the fixture includes a hole through which light is irradiated, and
the one or more images are an image obtained by photographing the tail upon irradiation of the light from the hole.

13. The tail vein injection system according to claim 1, wherein
the at least one processor is configured to
detect a pressure of a drug solution to be injected after the puncture, and
control an injection of the drug solution based on the detected pressure.

14. The tail vein injection system according to claim 13, wherein
the at least one processor is configured to
stop the injection of the drug solution upon the detected pressure exceeding a threshold.

15. The tail vein injection system according to claim 1, wherein
the at least one processor is configured to
determine whether or not the syringe needle is inserted into the tail vein based on a recognition result of blood in a syringe using an image.

16. The tail vein injection system according to claim 1, wherein
the at least one processor is configured to
adjust the position and the posture of the syringe needle using a position adjuster having five or more axial degrees of freedom.

17. The tail vein injection system according to claim 1, wherein
the at least one processor is configured to
calculate three-dimensional positions of two points on the tail vein based on two or more of the one or more images obtained by photographing the tail under conditions in which at least either or both of a photographing position and a photographing direction are different.

18. The tail vein injection system according to claim 17, wherein
the at least one processor is configured to
adjust the position and the posture of the syringe needle such that the syringe needle is inserted along a line passing through the two points.

19. The tail vein injection system according to claim 1, wherein
the at least one processor is configured to
obtain the one or more images using two or more cameras, and
the two or more cameras are a visible light camera.

20. The tail vein injection system according to claim 1, further comprising:
a retainer configured to retain a body of an animal having the tail, the retainer having a cylindrical shape that is open forward of the animal.

21. A tail vein injection method, comprising:
calculating, by at least one processor, information including a position of a tail vein based on one or more images of a tail;
adjusting, by the at least one processor, a position and a posture of a syringe needle based on the calculated information; and
after the adjusting, performing, by the at least one processor, a puncture of the tail vein using the syringe needle.

22. A non-transitory computer-readable storage medium storing a program that causes a computer to:
calculate information including a position of a tail vein based on one or more images of a tail;
adjust a position and a posture of a syringe needle based on the calculated information; and
after the adjusting, perform a puncture of the tail vein using the syringe needle.
